(19) United States
(12) Patent Application Publication
(10) Pub. No.: US 2003/0149341 A1
(43) Pub. Date: Aug. 7, 2003

(54) Retractor and/or distractor for anterior cervical fusion

(76) Inventor: Guy L. Clifton, Houston, TX (US)

Correspondence Address: Gilbreth & Associates, P.C., PO Box 2428, Bellaire, TX, 77402-2428, US

(21) Appl. No.: 10/068,221

(22) Filed: Feb. 6, 2002

(51) Int. Cl. A61B 1/32

(52) U.S. Cl. 600/210

(57) ABSTRACT

A surgical device having a blade with a surface suitable for abutting against tissue, and further having an anchor guide portion for receiving an anchor. The device further includes an anchor positioned through the anchor guide portion, having a first end suitable for anchoring into bone, and a second end suitable for engagement with a surgical distractor. In addition to being using during retraction, the device may be further used to distract without any repositioning of the device.

### BACKGROUND OF THE INVENTION

1. Field of the Invention

The present invention relates to methods and apparatus relating to surgery. In another aspect, the present invention relates to surgical instruments and methods of using surgical instruments. In even another aspect, the present invention relates to surgical retractors and to surgery using such surgical retractors. In still another aspect, the present invention relates to retractors for use during an anterior cervical fusion surgical procedure, and to methods of conducting an anterior cervical fusion surgical procedure.

2. Description of the Related Art

The human spine is subject to a number of ailments and afflictions, including “herniated discs.”

The spinal canal is like a tunnel which runs up and down the human spine. This canal sits directly behind the bony blocks which make up the spine (vertebrae) and contains the nerves (spinal cord and nerve roots) running from the brain to all areas of the body. When something causes a narrowing of this canal then the nerves can become irritated or squeezed. This can lead to a variety of symptoms ranging from tingling, numbness, and weakness to severe pain and paralysis.

A herniated disc (often called a slipped disc) is one of several common conditions which can narrow the spinal canal, and occurs when a fragment of the disc nucleus is pushed out of the outer disc margin, into the spinal canal through a tear or “rupture.” The spinal canal has limited space which is inadequate for the spinal nerve and the displaced herniated disc fragment. In the herniated disc's new position, it presses on spinal nerves, producing pain down the accompanying leg or arm. This may produce sharp, severe pain down the entire leg and into the foot or into the arm. The compression and subsequent inflammation is directly responsible for the pain one feels down the leg, termed “sciatica.” The direct compression of the nerve may produce weakness in the leg or foot or arm in a specific pattern, depending upon which spinal nerve is compressed.

One solution for such a condition is the surgical removal of the herniated disc(s). If the herniated disc(s) are in the neck of a human, the surgical procedure is known as anterior cervical fusion, and requires retraction of the pharynx and the carotid sheath and its contents. The herniated disc(s) is(are) removed, and bone graft(s) seated in place of the herniated disc(s).

Of course, the fragile nature of the pharynx, esophagus and the carotid sheath are well known, and much care must be taken to not cause undue damage to the pharynx or esophagus, and to not pierce the carotid sheath or pharynx.

Retraction of the pharynx and the carotid sheath and its contents is accomplished by use of a retractor, which may generally be described as 2 narrow blades attached to a device with arms which spreads the 2 blades apart laterally (i.e., perpendicularly) across the line of the spine and holds them. The scalpel and the drill are not the only instruments posing a danger to the pharynx and the esophagus. The retractor may also pose a danger if not properly designed.

While there are a number of designs in commercial use, they generally all utilize a lateral blade that is toothed and grips the longus coli, and a rather smooth medial blade that cannot be toothed because of danger of injuring the soft pharynx and esophagus.

The main design weakness of the known retractors is that the pharynx will tend to slide along, and ultimately under the medial blade, resulting in the need to constantly rearrange and reset the blades during anterior cervical fusion, perhaps as often as every 10 minutes during the retraction. Each rearrangement and resetting of the blades distracts the surgeon's attention, risks damage to the pharynx and esophagus, requires another several minutes of extra time. A typical surgery might require anywhere from at least 3 to 10 rearrangements and resettings of the blades for a skilled surgeon, adding time to the procedure.

The is another problem encountered when two sets of retractor arms are used. While the inferior set rests flat against the chest causing no problem, the superior set rests against the jaw and projects out beyond the jaw so that the blades are torqued. As an embodiment of this invention, the use of an arm which holds two blades allows the use of only the inferior set when two sets of blades are used, and permits the use of 3-4 sets of blades simultaneously.

Once the herniated disc(s) is(are) removed, the vertebral body must be spread apart in order to seat the bone graft(s). A secondary design weakness of the known retractors is that the vertebral body must be distracted (i.e., spread apart longitudinally along the line of the spine) in a separate maneuver with separate instruments in order to seat the bone graft(s).

There have been a number of patents related to surgical retractors.

U.S. Pat. No. 1,252,177, issued Jan. 1, 1918, to J. B. Redfield, discloses a mouth gag.

U.S. Pat. No. 569,839, issued Oct. 20, 1896, to J. T. R. Roeloffs, discloses a retractor.

U.S. Pat. No. 1,550,403, issued Aug. 18, 1925, to E. N. Turkus, discloses a retractor for amputation purposes.

U.S. Pat. No. 1,618,261, issued Feb. 22, 1927, to H. J. Arbogast, discloses a viscera retainer for use in abdominal operations.

U.S. Pat. No. 2,075,534, issued Mar. 30, 1937, to C. V. McCormack, discloses a mouth prop.

U.S. Pat. No. 2,238,563, issued Apr. 15, 1941, to W. H. Jacques, discloses an embalming instrument.

U.S. Pat. No. 2,541,516, issued Feb. 13, 1951, to H. S. Ivory, et al, discloses a retractor means for surgical use.

U.S. Pat. No. 2,751,903, issued Jun. 26, 1956, to H. S. Ivory, et al, discloses an adjustable retractor for surgical use.

U.S. Pat. No. 3,038,467, issued Jun. 12, 1962, to E. J. Sovatkin, discloses a surgical instrument.

U.S. Pat. No. 3,176,682, issued Apr. 6, 1965, to D. J. Wexler, discloses a self-retaining vaginal surgical retractor.

U.S. Pat. No. 3,278,207, issued Oct. 11, 1966, to E. Z. Barish, et al, discloses a ball joint structure.

U.S. Pat. No. 3,384,078, issued May 21, 1968, to W. K. Gauthier, discloses a surgical apparatus including an adjustable retractor blade secured to a mounting means. The blade has a transversely corrugated portion or arm adjustable secured to the mounting means and a right angular extending support portion with spaced parallel ribs along its lateral edges. A second portion is adjustably secured to the support portion by means of reversely bent resilient side edge portions or flanges. These edge portions have recesses which mesh with the ribs on the support member to hold the adjustable blade portion in position.

U.S. Pat. No. 3,463,144, issued Aug. 26, 1969, to Daniel O. Hammond, discloses an upper abdominal retractor comprising amounting arm adjustably mountable in a retractor frame and a retractor blade having a substantially flat central section depending from the mounting arm at an angle of about 90§ and two substantially flat wings extending laterally from said central section, the ends of said wings being free and spaced apart to define a notch between said wings.

U.S. Pat. No. 3,565,061, issued Feb. 23, 1971, to Verne J. Reynolds, discloses two cooperating members, a lower member and an upper member, both made of suitable plastic material. The lower member has a concave upwardly facing arcuate handle portion of a radius of about 4 inches. At its upper end is an integral shank of about twice the width of the handle and concave in cross section with the concave portion facing upwardly and projecting on out from the end of the shank portion in an arcuate right angle. This projecting end portion is a tongue portion that is bifurcated and also concave and facing upwardly. The handle of the lower member has sides that extend up and inwardly and form a channel within which is slidingly received the handle of a correspondingly shaped upper member having like parts except that the shank at the upper end of the handle is longer and has an elongated relatively large aperture there through providing a view and access for preparation instruments and between the spaced apart tongues. The upper member shank is concave facing downwardly as is its bifurcated projecting tongue. The lower member handle has an integral downwardly extending finger engaging abutment at its lower end. The upper member handle has an integral curved upwardly thumb engaging abutment at its lower end.

U.S. Pat. No. 3,638,973, issued Feb. 1, 1972, to Charles E. Poletti, discloses a joint means for coupling two elements forming part of a work-supporting arm wherein the joint means is operable.

U.S. Pat. No. 3,729,006, issued Apr. 24, 1973, to Joseph R.Wilder, discloses a hand-held surgical retractor fabricated of resilient plastic material, the retractor being light-weight, glare-free and functionally superior to existing metallic instruments. The retractor includes a non-skid blade whose inner surface is slightly concave, the blade having an array of circular apertures therein whereby when the blade is pressed against tissue, the tissue bellies into the blade to provide a contact there between which resists displacement even when low pulling forces are exerted on the retractor.

U.S. Pat. No. 3,731,673, issued May 8, 1973, to William X. Halloran, discloses a self-retaining muscle retractor including a pair of elongated members formed on their respective one extremities with bone-engaging portions and on their respective opposite extremities with lever arms. One of the lever arms has one end of a rigid brace pivotally connected thereto and the free end of such brace is engageable with a latching element included on the other lever arm whereby the bone-engaging portions may be inserted in an incision and engaged on opposite sides of a bone, the lever arms spread apart to retract the muscles away from such bone and the free end of the brace engaged with the latching element to maintain the incision open for convenient access to the bone.

U.S. Pat. No. 3,858,578, issued Jan. 7, 1975, to Simcha Milo, discloses a device for holding surgical instruments firmly in place. A surgical instrument is attached to a retaining arm, the rigidity of which is controlled by a fluid actuated operating apparatus. By activating the operating apparatus, the arm can be made flexible as the instrument is being positioned and rigid once the instrument is in place.

U.S. Pat. No. 4,116,232, issued Sep. 26, 1978, to, discloses a surgical retractor according to the invention comprises a straight shaft, a blade and means for pivotally connecting the blade to the shaft with the blade oriented perpendicularly to the shaft and being free to turn about an axis perpendicular to the shaft. The retractor may further comprise means for telescopically adjusting the length of the shaft and releasably fixing the shaft at the adjusted length. A sole may be formed on the free extremity of the blade most remote from the pivotal connection, the sole being symmetrically formed on the blade and defining surfaces in planes perpendicular to the turning axis of the blade, the surfaces extending equally beyond the thickness of the blade in opposite directions.

U.S. Pat. No. 4,616,635, issued Oct. 14, 1986, to Wolfhard Caspar, discloses an instrument for the splaying of the edges of a wound which has at least one blade. The blade has a middle part, a rim projecting at right angles from the middle part, and a mount on which the middle part is held. The middle part has two parts movable against each other by an actuator comprising a turnable rod with a screw thread section.

U.S. Pat. No. 4,738,248, issued Apr. 19, 1988, to Charles D. Ray, discloses a surgical retractor can be used in conjunction with an electro-surgical instrument without ensuring against the two coming too close together or touching when the surface of the tip of the retractor is electrically and thermally nonconductive and has a Rockwell C hardness of at least 55. The tip of the retractor may have a metal core which is provided with a hard, electrically insulating surface by sintering a ceramic such as aluminum oxide or zirconium oxide. Instead, the tip can be a piece of ceramic which is mounted onto a broad metal band by means of a short length of metal tubing.

U.S. Pat. No. 5,271,384, issued Dec. 21, 1993, to James A. McEwen, discloses an apparatus useful in surgery for holding surgical instruments such as retractor blades in a number of different positions required by a surgeon for the performance of a surgical procedure, including an actuation means responsive to a control signal, a signal generating means which enables an operator to generate the control signal and which transmits the control signal to the actuation means, for causing the actuation means to move the surgical instrument.

U.S. Pat. No. 5,363,841, issued Nov. 15, 1994, to Wesley L. Coker, discloses a retractor of the present invention has supporting arms connected at an angle to the retractor blades to remove the arms of the retractor from the top edges of the wound and thus allow for a greater amount of latitude in applying various mechanical devices to the spine. The angled arms on the retractor blades themselves placed these structures deep within the wound and thus apply distraction forces where they are needed the most—near the spine itself to spread the muscles away from the spine and make it easier to see. The blades of this spinal retractor apply distraction forces deep within the wound where they are needed the most. On either side of the angled arms deep within the wound are vertical fingers projecting deeper into the wound on the lower portion and up out of the wound on the upper portion. The distances between the fingers provide further lateral angulation of instruments used in the wound itself for the placement of the spinal fixation devices. This retractor also has a laterally projecting anchor peg extending from the muscle side of the retractor blade which is meant to lie beneath the dorsolumbar fascia. This anchor peg locks the retractor into the depths of the wound and prevents its migration up and out of the wound as is so frequently encountered in other types of spinal retractors.

U.S. Pat. No. 5,512,038, issued Apr. 30, 1996, to Darrell D. O'Neal, discloses a surgical retraction apparatus having a retractor blade with a curved blade and a quick release mechanism. The blade has a complex curved shape and notches on the sides to reduce tissue damage during retraction. Blades can be removed from a rack and pinion mechanism by sliding the bushing of the blade over the rack arm.

U.S. Pat. No. 5,529,571, issued Jun. 25, 1996, to Elie C. Daniel, discloses a surgical instrument of the invention consists of a pair of arms hinged together at an intermediate point for relative pivoting movement. The arms are provided with obliquely oriented handles at one end thereof. The opposite ends of the arms support removable posts having means for removably securing a plurality of different retractor or compressor heads thereto. The posts are pivotably connected to the arms to allow the posts to achieve a variety of orientations relative to the arms. A variety of retractor and compressor heads and different shaped posts are provided to allow the instrument to be used in a wide variety of surgical procedures. Separate serrated locking mechanisms are provided to lock the arms in either a retraction or compression position. In one embodiment the posts are secured to one another such that they undergo parallel retraction/compression.

U.S. Pat. No. 5,618,260, issued Apr. 8, 1997, to Wolfhard Caspar, discloses a surgical instrument in order to make the covered tissue areas visible in an X-ray picture when using a surgical instrument for the retention of tissue which has at least one valve held on one side, also when using a material impermeable to X-rays, it is suggested that the contact surface of the valves have openings in a distribution which leaves an increasing, non-perforated cross-sectional area from the free end of the contact surface up to its holder.

U.S. Pat. No. 5,728,046, issued Mar. 17, 1998, to Heinz M. Mayer, discloses in a surgical retractor having a frame on which at least two retractor elements are mounted, if desired, so as to be displaceable, to enable individual adjustment of the forces exerted by the retractor elements on the surrounding tissue, it is proposed that the frame have at least one support foot which carries attachment means for fixing on a bone.

U.S. Pat. No. 5,931,777, issued Aug. 3, 1999, to Gerard A. Save, discloses a tissue retractor with particular use in spinal surgery comprises a pair of pivotally linked arms, each with a blade mounted thereto by a ball and socket joint to allow the blades free movement relative to the arms. The blades have sharp piercing tips to anchor to bone, and allow the blades to pivot around the position of the piercing tips. The '777 patent retractor is stated to rely on pivoting movement of the blades around an anchoring pin to displace tissues. Certainly, with these sharp tips, no competent surgeon would use these in the neck for fear of pharyngeal damage. The '777 patent retractor is operable by placing the blades in a wound opening; securing the sharp tips to a portion of bone at positions a distance apart from each other to define a desired surgical field; and operating the retractor to cause the blades to separate and to retract tissues surrounding said wound opening by outward pivoting of the blades relative to the position of the sharp tips. A supplemental retractor blade or blades may be provided by a linking armature for additional retraction generally perpendicularly to a retraction action provided by the main retractor blades. While this blade is

However, in spite of these advancements in the prior art, none of these prior art references disclose or suggest a surgical retractor that can safely hold the pharynx in a retracted position, and that can optionally be useful in the maneuver of spreading the vertebral body apart.

Thus, there is still a need for surgical retractors.

There is another need in the art for a surgical retractor that can safely hold the pharynx in a retracted position.

There is even another need in the art for a surgical retractor that can safely hold the pharynx in a retracted position, and that can optionally be useful in the maneuver of spreading the vertebral body apart.

There is still another need in the art for a surgical retractor that can be utilized without projecting against the jaw and being under torque.

These and other needs in the art will become apparent to those of skill in the art upon review of this specification, including its drawings and claims.

### SUMMARY OF THE INVENTION

It is an object of the present invention to provide for surgical retractors.

It is another object of the present invention to provide for a surgical retractor that can safely hold the pharynx in a retracted position.

It is even another object of the present invention to provide for a surgical retractor that can safely hold the pharynx in a retracted position, and that can optionally be useful in the maneuver of spreading the vertebral body apart.

It is still another object of the present invention to provide for a surgical retractor that can be utilized without projecting against the jaw and being under torque.

These and other objects of the present invention will become apparent to those of skill in the art upon review of this specification, including its drawings and claims.

### BRIEF DESCRIPTION OF THE DRAWINGS

In the FIGs., like reference numbers refer to like structures. Additionally, both within a drawing, and between drawings, similar looking structures can be considered to be similar (whether marked by a reference number or not).

FIGS. 1, 2 and 3 are, respectively, an isometric view, top view, and a side cutaway view cut away at 5-5 of FIG. 2, of the single anchor per blade embodiment of a surgical apparatus 10 and method of the present invention (shown positioned across two vertebra of vertebrae 11) comprising a retractor 200, anchored blade 100 and second blade 500.

FIGS. 4, 5, and 6A are, respectively, an isometric view, top view, and a side cutaway view cut away at 6-6 of FIG. 5, of the double anchor per blade-interlocking blade embodiment of a surgical apparatus 10 and method of the present invention (shown positioned across three vertebra of vertebrae 301) comprising a retractor 200, anchored blade 100 and second blade 500. FIG. 6B is a detail from FIG. 6A.

FIGS. 7 and 8 are, respectively, an isometric view and top view, of the single anchor per blade embodiment of a surgical apparatus 10 and method of the present invention (shown positioned across three vertebra of vertebrae 301) comprising a retractor 200, anchored blade 100 and second blade 500.

FIGS. 9, 10 and 11 are, respectively, an isometric view, top view, and a side cutaway view cut away at 11-11 of FIG. 10, of the double anchor per blade embodiment of a surgical apparatus 10 and method of the present invention (shown positioned across two vertebra of vertebrae 301) comprising a retractor 200, anchored blade 100 and second blade 500.

FIGS. 12, 13 and 14, there are shown the same views as in FIGS. 1, 2 and 3, respectively, except that, in FIGS. 12, 13 and 14, a distractor 55 has been connected to anchors 601.

### DETAILED DESCRIPTION OF THE INVENTION

One aspect of the present invention relates to an anchorable blade useful in surgical procedures with a retractor.

Another aspect of the present invention, relates to a surgical instrument comprising a surgical retractor and the anchorable blade, which instrument is especially useful for conducting spinal surgery where it is necessary to displace the soft delicate tissues anterior of the spine to allow access to the spinal vertebrae. Of course, this anchorable blade and/or resulting surgical instrument may be used in other applications where it is desired to displace tissues adjacent to bone matter in the body.

The surgical instrument of the present invention includes a retractor, an anchorable blade and removable anchors, and a second blade that is complimentary to the anchorable blade.

The surgical instrument of the present invention may be utilized during spinal surgery, such as for example, an anterior cervical fusion, to retract the pharynx or the carotid sheath and its contents. As discussed in the background above, the fragile nature of the pharynx, esophagus and the carotid sheath are well known, and much care must be taken to not cause undue damage to the pharynx or esophagus, and to not pierce the carotid sheath.

Thus, when conducting an anterior cervical fusion with the surgical instrument of the present invention, the second blade is utilized as the lateral blade and will be facing the longus coli, and the anchorable blade is utilized as the medial blade and will be facing the soft pharynx and esophagus.

Like any retractor, the present invention utilizes complimentary pairs of blades. As used herein, “complimentary” merely means a pair of retractor blades that together are used to retract tissue, and the blades may or may not be the same. With the present invention, the complimentary second or lateral blade may be any suitable conventional or nonconventional blade that keeps the longus coli out of the surgical field. This second blade may also be an anchorable blade. It is generally believed that any of the commercially available blades typically utilized for retracting the longus coli may be utilized as the second or lateral blade. These commercially available blades generally include a toothed or otherwise gripping edge which grips the longus coli to prevent it from slipping into the surgical field.

The anchorable blade of the present invention will include a non-damaging edge for facing the soft delicate pharynx, esophagus and carotid sheath. The “non-damaging” edge should be suitable for manipulation around and incidental contact with the soft delicate pharynx and esophagus, such that any damage to those tissue are minimal. For example, the non-damaging edge could be a smooth-surfaced edge. As a further example, the non-damaging edge should lack any sharp or pointed portions that could cut or pierce the soft delicate tissue. The anchorable blade will include passages, notches, slots or the like, for receiving removable anchors, such as pins, screws, pegs, rods, fasteners, and the like, which are utilized to anchor the anchorable blade to vertebrae. Of course, such an anchor could also be incorporated into the blade.

While any suitable anchors may be utilized, it is preferred that removable anchors be utilized. Removable anchors are removable from the blade to allow retraction to be conducted without the anchors in place to reduce risk of damage to tissue. Once the anchorable blade has been retracted to its desired location, the anchorable blade is then anchored to the vertebrae to prevent tissue from slipping under the removable blade and into the surgical field.

In many instances it may also be necessary or desired to distract the vertebrae utilizing the anchored blades of the present invention.

Unlike prior art devices which are either retraction or distraction devices, the present invention can be utilized to retract tissue, and then once in place may be utilized to distract the vertebrae. The methods and apparatus of the present invention will now be described by reference to the figures.

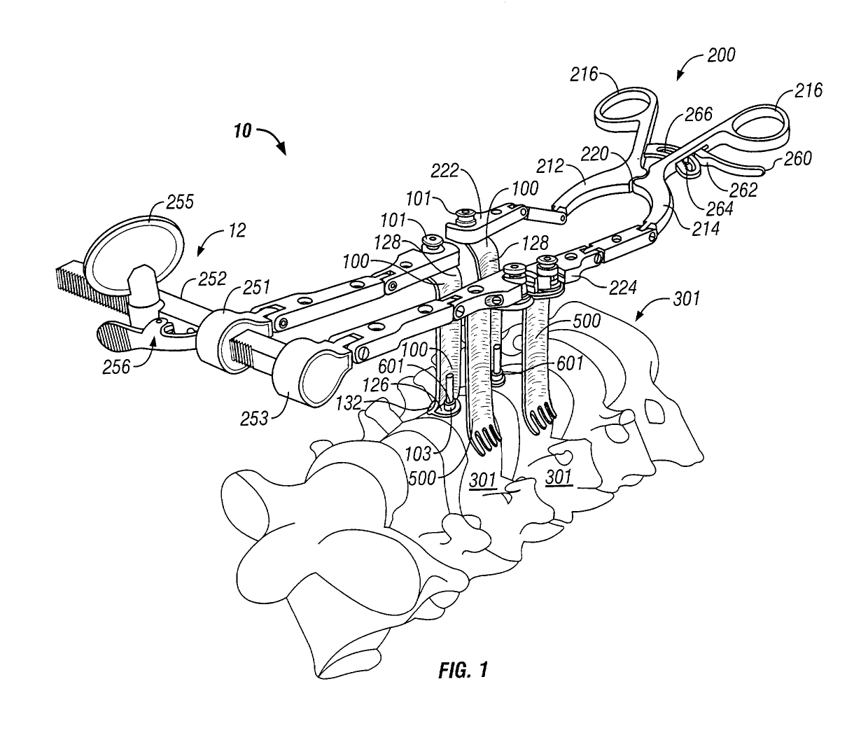

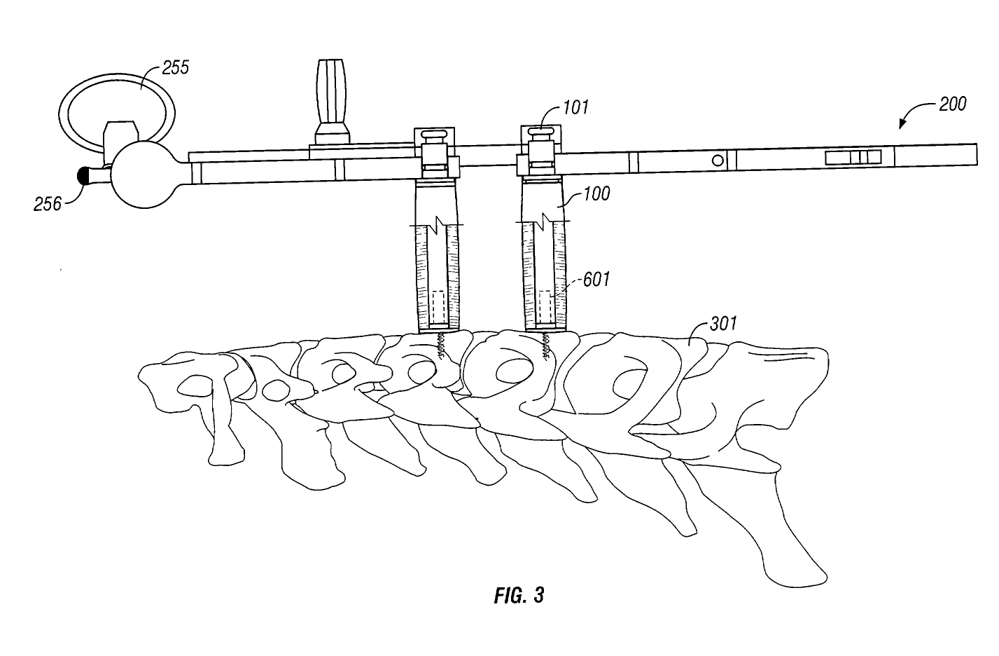

Referring first to FIGS. 1, 2 and 3, there are shown, respectively, an isometric view, top view, and a side cutaway view cut away at 5-5 of FIG. 1, of the single anchor per blade embodiment of a surgical apparatus 10 and method of the present invention (shown positioned across two vertebra of vertebrae 301) comprising a retractor 200, anchored blade 100 and second blade 500.

It should be understood that any suitable retractor may be utilized in the present invention, provided it is operable with anchored blade 100 and second blade 500. As a non-limiting example, retractor 200 of FIG. 1 is a Cloward-style cervical retractor, commercially available from Ruggles Instruments.

Retractor 200 generally comprises a pair of arms 212 and 214, which each have finger grip loops 216 at proximal ends 218 thereof as are conventional in retractor design. A pivotal connection 220 connects the pair of arms 212 and 214 at mid-portion thereof. This pivotal connection 220 may comprise any suitable type of connection, as a common example, pivot pins on one arm 212 received in pivot pin holes on the other arm 214. Anchorable blade 100 is mounted to arm 212 at distal ends 222 thereof, and second blade 500 is mounted to arm 214 at distal ends 224 thereof.

The retractor of the present invention is optionally preferably provided means for locking the arms 212 and 214 in a fixed relationship, non-limiting examples of which include any of the well known various ratchet mechanisms or screw tightened elements. As a non-limiting specific example, shown in FIG. 1 is a simple locking ratchet mechanism wherein a thumb lever 260 with teeth 262 engages a curved/ ratchet bar 264 with mating teeth 266.

While retractor 200 is shown as having slots at distal ends 222 and 224 for receiving blades 100 and 500, it should be understood that any suitable mechanism and method may be utilized to engage blades 100 and 500, including those as shown in U.S. Pat. No. 5,931,777, issued Aug. 3, 1999 to Sava, herein incorporated by reference. Retractor 12 is a suitable alternative retractor, in which retractor arms 251 and 253 are maintained a desired distance apart along rod 252, by locking and adjusting mechanism 255 and 256.

Operation of retractor 200 by squeezing finger grip loops 216 together causes distal ends 222 and 224 and the blades 100 and 500 mounted thereto to separate apart from each other, to thereby displace tissue as further described below. Thumb lever 260 is operated to lock arms 212 and 214 in a fixed relationship.

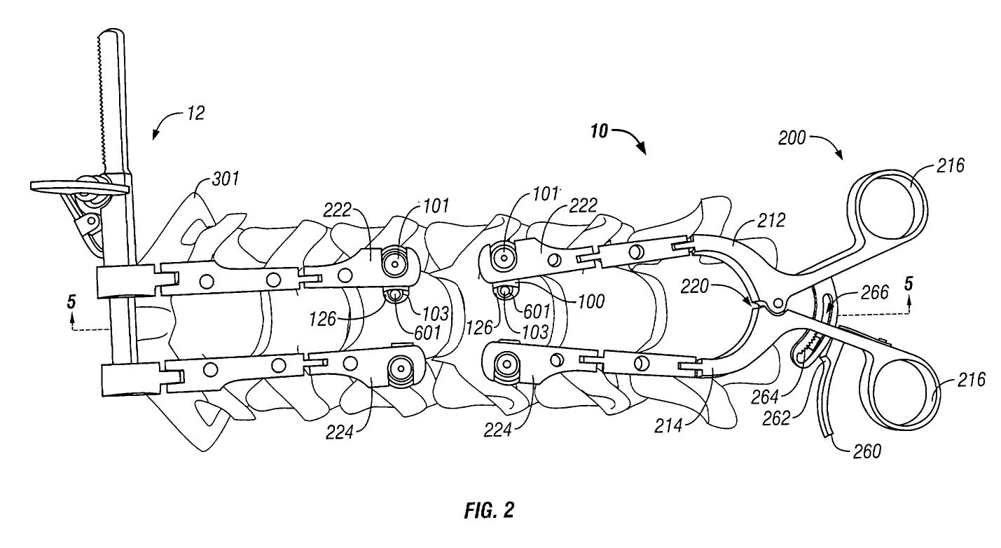

Blade 100 may be any suitable shape or dimension as required for surgery. In the embodiment as shown in FIGS. 1 and 2, blade 100 includes a connector 101 that engages distal end 222. Blade body 128 serves to extend down into the wound to vertebra 11 and keep retractor 200 suitably positioned thereabove. At the far end of blade 100 is retaining edge 132 and anchor section 126.

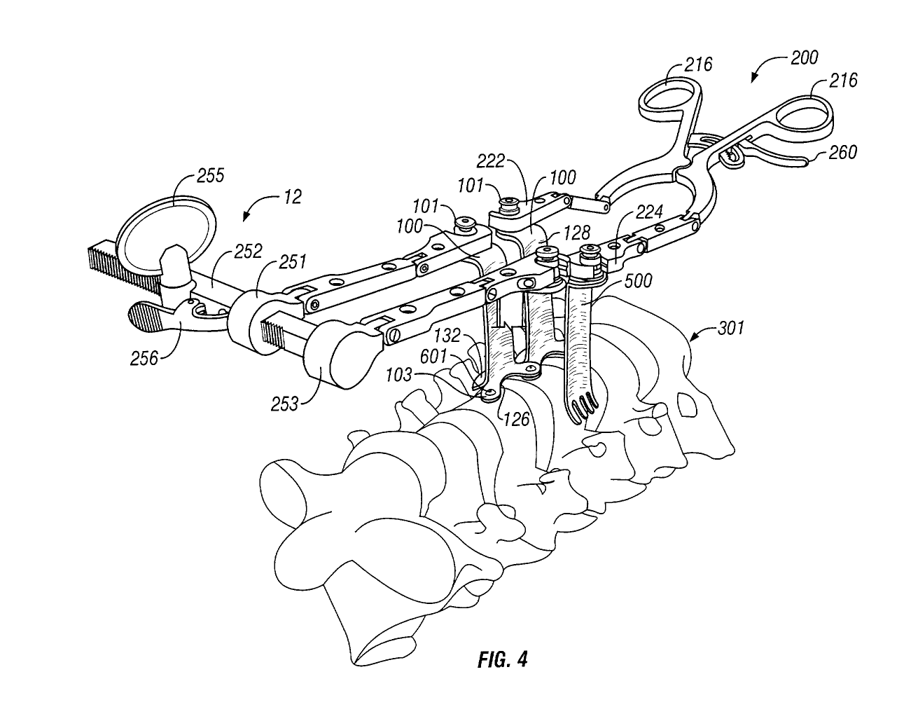

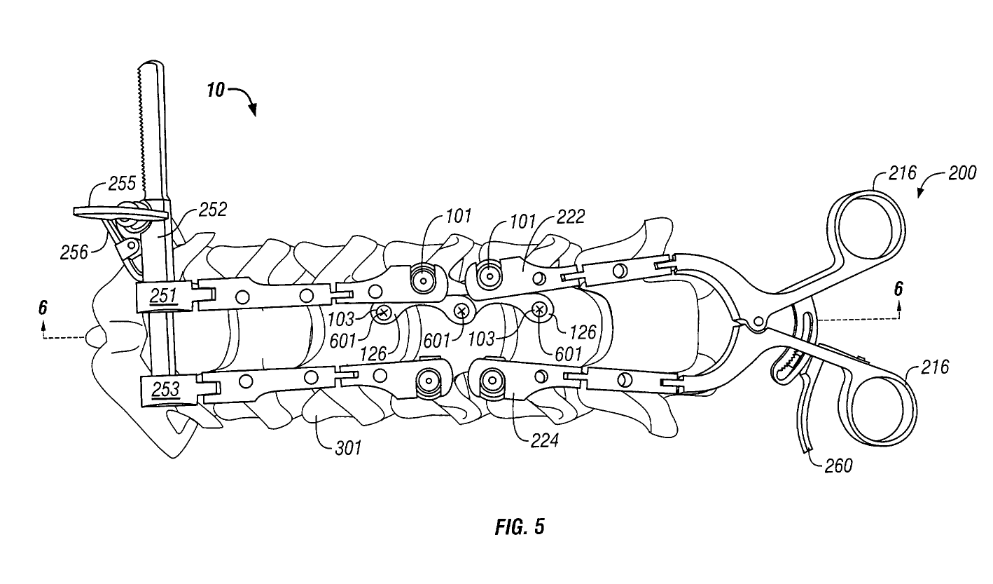

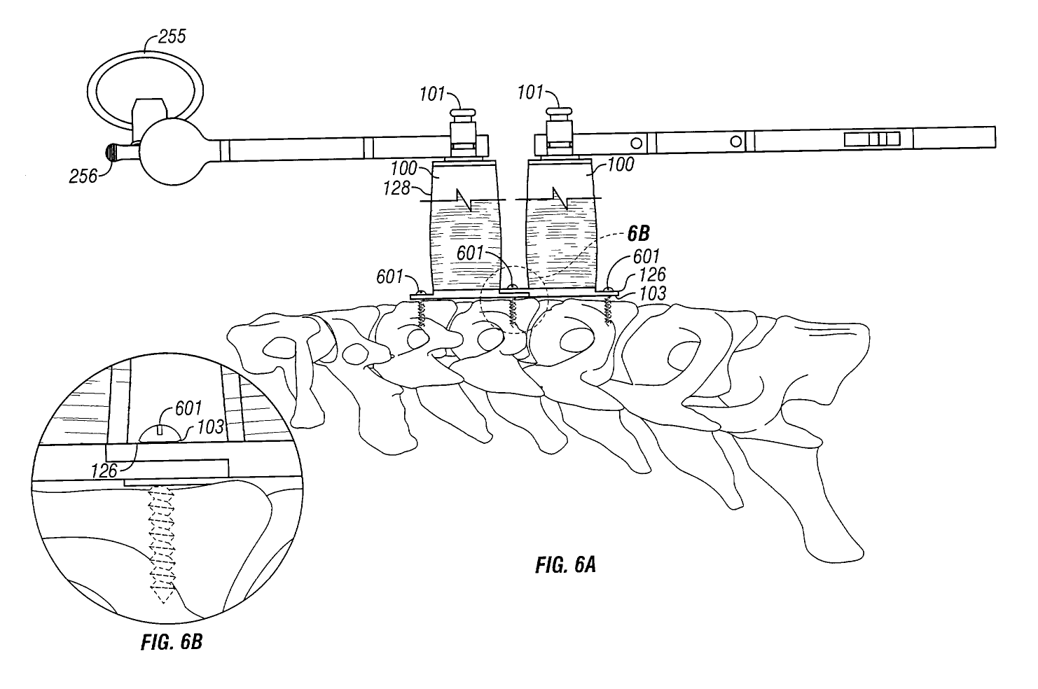

Anchor section 126 includes anchor receiving sections 103, which are generally holes, slots, grooves, or passages. Generally, one of the anchor receiving sections will be positioned on a first vertebrae, and the other anchor receiving section will be positioned on a second vertebrae. To accommodate allowing anchor blades 100 to abut against each other (across several vertebrae), anchor section 126 may be made to interengage or interlock with other anchor sections 126 (see FIGS. 4, 5 and 6 discussed below). As shown in FIGS. 4, 5, and 6, each of blades 100 accommodate two anchors, and interengage or interlock by sharing an anchor 601. Anchorable blade 100 has been anchored to vertebrae 301 by anchors 601 positioned through slots 103 and into vertebrae 301. While it may be possible to utilize tapered or threaded anchors 601 which are simply rotated into vertebrae 301, it is preferred to first drill pilot holes into vertebrae 301 and then rotate anchors 601 into the pilot holes.

In a preferred embodiment, anchors 601 will have a long body that extends upwardly away from the vertebrae to allow for attachment of a distraction device, so that distraction may be subsequently conducted.

Optionally, to minimize any movement of anchorable blade 100 which is being anchored against vertebrae, the bottom portion of anchor section 126 which is in contact with the vertebrae may be a surface that provides some amount of slip resistance. This can be provided, for example, by providing ridges or teeth on the surface, by roughening the surface, by coating the surface with or making the surface from a slip resistant material, or by providing a slip resistance material therebetween during surgery (i.e., something like a glue, or a something solid like a pad).

According to the present invention, the various portions of blade 100 may have same or different widths, to increase the size of the surgical field and/or the amount of tissue displaced, without increasing the size of components that might interfere with the surgeon's work.

Once anchored blade 100 is anchored to vertebrae 301, cartilage 302 is removed. Upon removal of the cartilage, the vertebra adjacent the removed cartilage will tend to move toward each other to fill the void left by the removed cartilage. Traditional retractor systems would allow vertebrae 301 to move toward each other, meaning that seating a bone graft requires that the vertebrae that have moved together be spread apart in a separate maneuver with separate instruments.

As another advantage of the present invention, anchored blade 100 can be utilized to keeps vertebrae spread apart a distance sufficient to allow for insertion of a bone graft (or other spacing member), without the need to engage separate instruments.

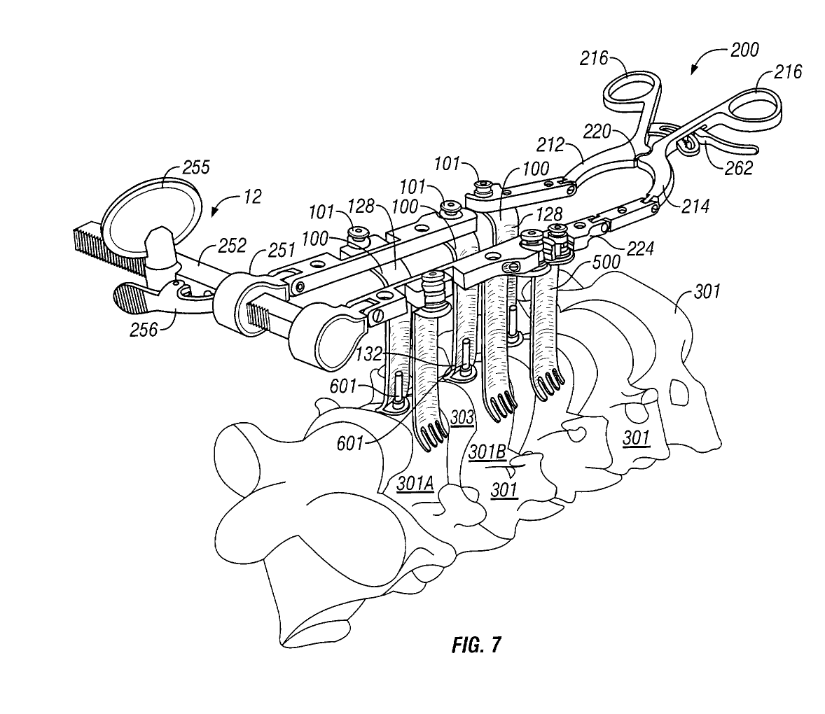

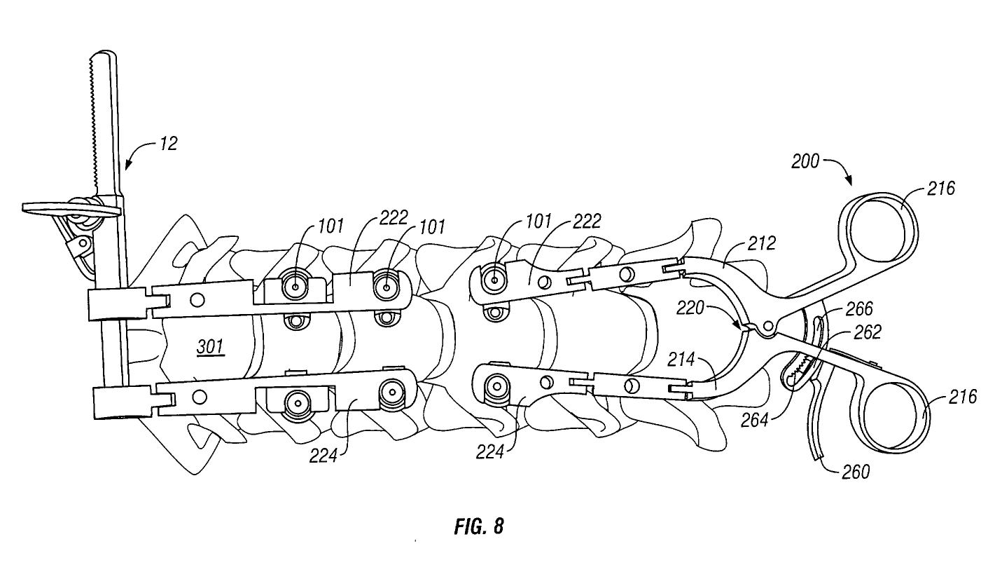

One way of maintaining separation of vertebra is by use of a pair of blades on a retractor, such that the blades do not move relatively to each other along the line of the vertebra. To illustrate, refer now to FIGS. 7 and 8, where there are shown, respectively, an isometric view and top view, of the single anchor per blade embodiment of a surgical apparatus 10 and method of the present invention (shown positioned across three vertebra of vertebrae 301) comprising a retractor 200, anchored blade 100 and second blade 500. Retraction with the present invention can be made across, 1, 2, 3 or more vertebra as desired. While retraction across 3 vertebra is shown with 1 pair of blades on one retractor, and 2 pair of blades across the other retractor, it should be understood that one retractor could be utilized to retract with 3 (or more) pair of blades.

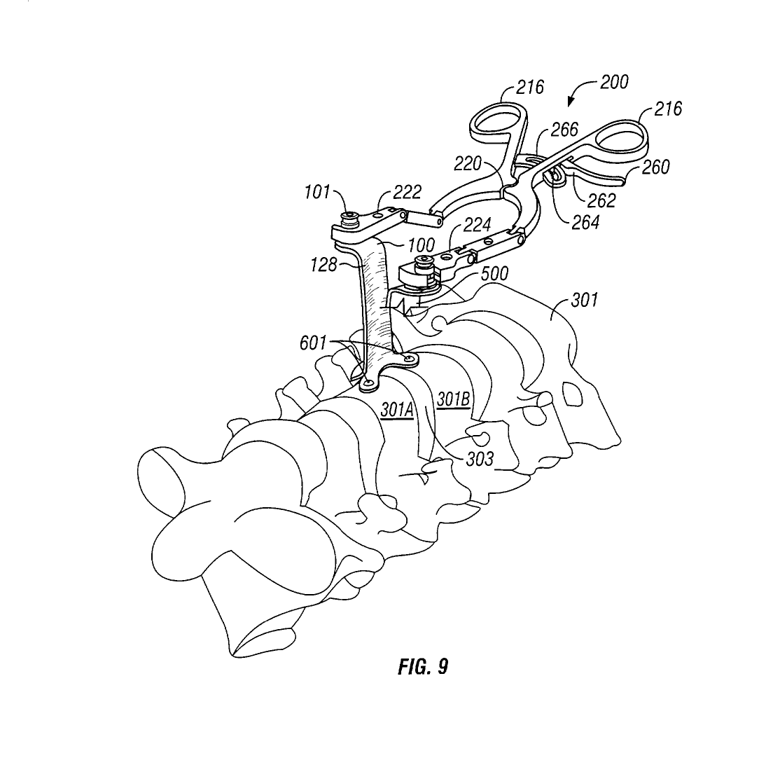

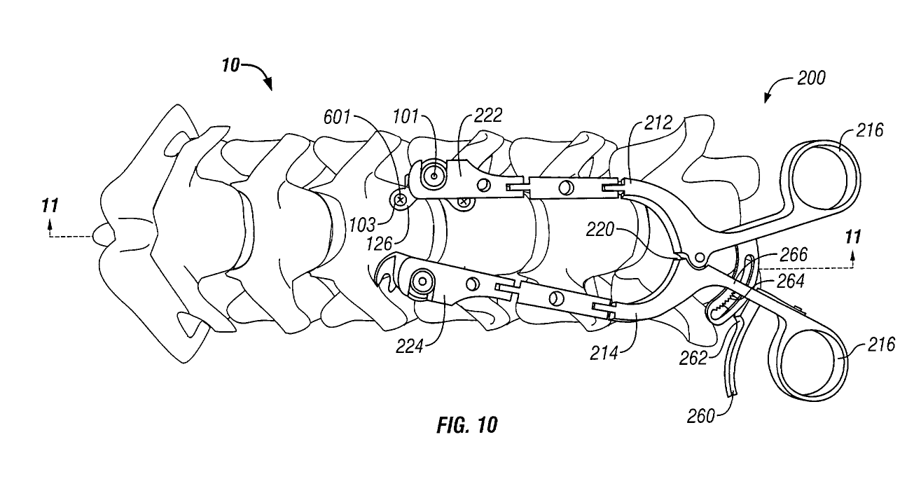

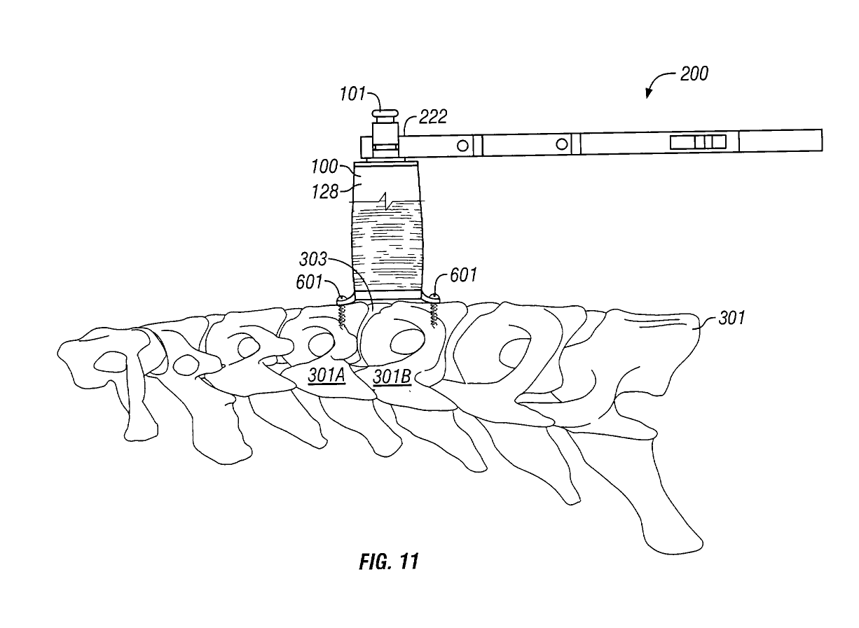

Another way of maintaining separation of vertebra is by use of two anchors on a blade, with one anchor in each of adjacent vertebra. To illustrate, refer now to FIGS. 9, 10 and 11, where there are shown an isometric view, top view, and a side cutaway view cut away at 11-11 of FIG. 10, of the double anchor per blade embodiment of a surgical apparatus 10 and method of the present invention (shown positioned across two vertebra of vertebrae 301) comprising a retractor 200, anchored blade 100 and second blade 500. Notice that even with removal of cartilage 303 from between vertebra 301A and 301B, those vertebra cannot move relative to each other along the line of vertebra 301, because blades are anchored to the vertebra and to retractor 12.

According to the present invention, a number of anchored blades 100 may be utilized to remove a plurality of cartilages 302. As an illustration, see the embodiment as shown in FIGS. 7 and 8 discussed above. As another illustration, refering now to FIGS. 4, 5 and 6, in which there are shown an isometric view, top view, and a side cutaway view cut away at 14-14 of FIG. 10, of the double anchor per blade-interlocking blade embodiment of a surgical apparatus 10 and method of the present invention (shown positioned across three vertebra of vertebrae 301) comprising a retractor 200, anchored blade 100 and second blade 500. Like the embodiment shown in FIGS. 9, 10 and 11, notice that even with removal of cartilage 303 from between vertebra 301A and 301B, those vertebra cannot move relative to each other along the line of vertebra 301, because each is anchored to blade 100. It is possible that vertebra 301A and 301B might be able to slightly close the missing cartilage gap, because blades 500 are not anchored to the vertebra, it should still be possible to insert a bone graft or other spacing member by the gap that is maintained by blade 100.

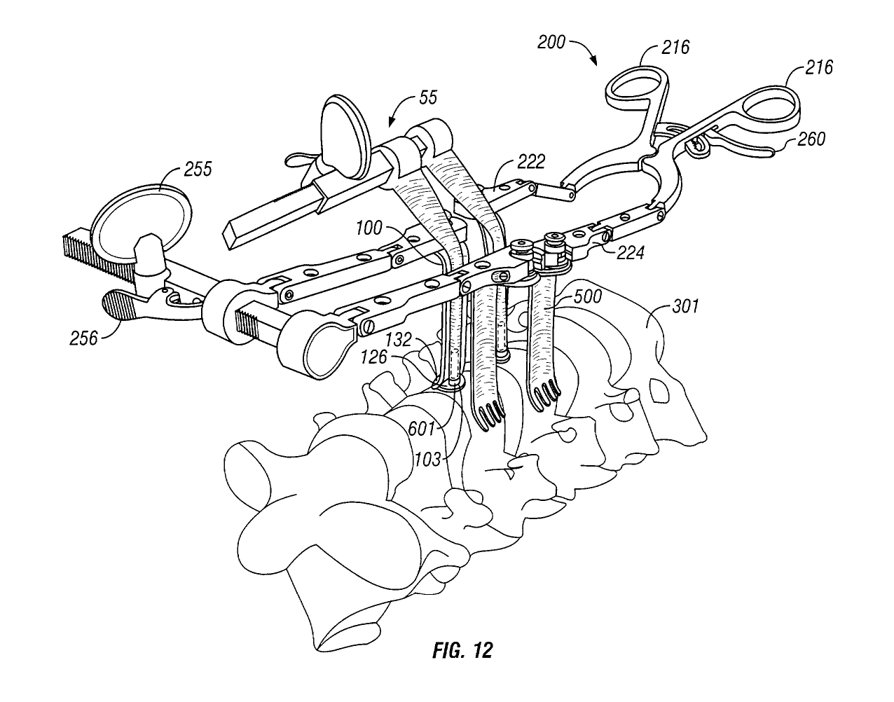

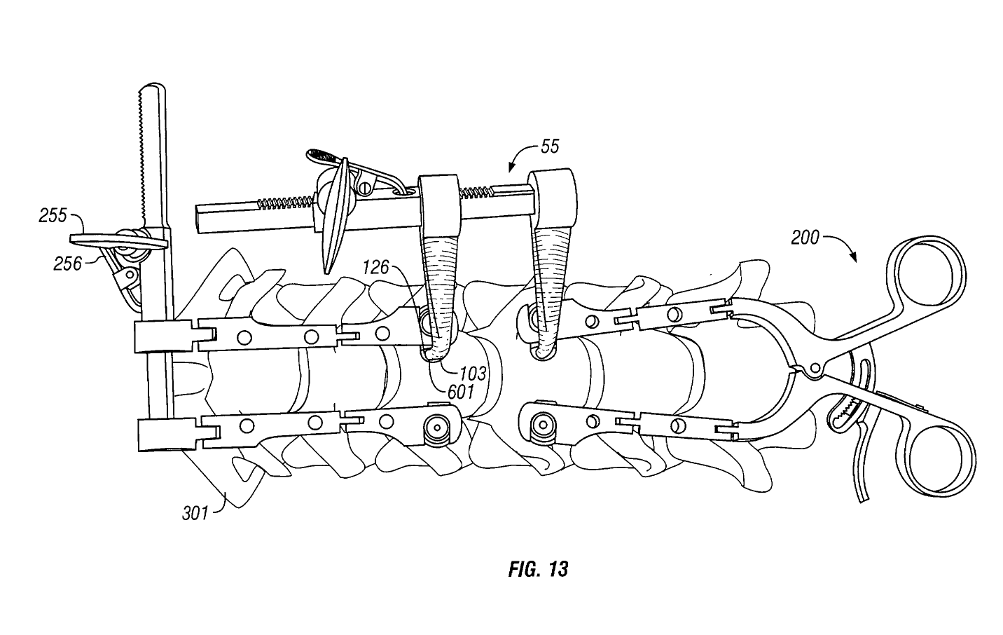

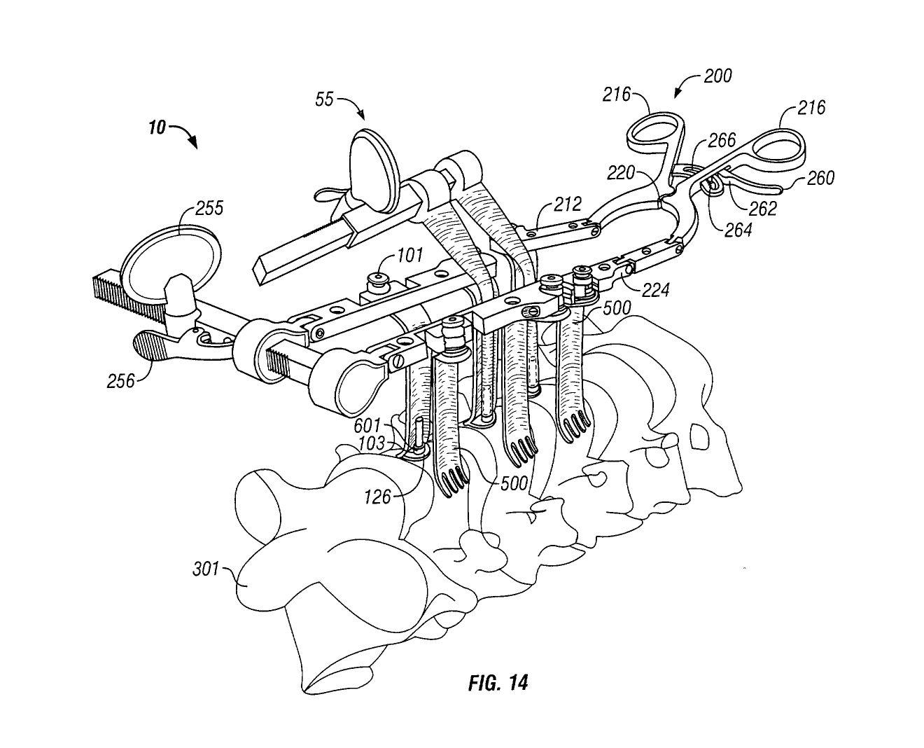

The present invention may also be used in the distraction of vertebrae. For example, referring now to FIGS. 12, 13 and 14, there are shown the same views as in FIGS. 1, 2 and 6, respectively, except that, in FIGS. 12, 13 and 14, a distractor 55 has been connected to anchors 601. This embodiment of blades 100 employs a centrally positioned anchor 601. In operation, retraction would first be accomplished as described above affixing anchors 100 in place with anchors 601. To distract, the surgeon then attaches a distractor to anchors 601, and pulls vertebrae 301 apart along the line of the vertebra.

All references, articles, patent applications and patents referred to herein, are hereby incorporated by reference.

While the methods and apparatus of the present invention have been illustrated mainly by reference to an anterior cervical fusion, it should be understood that the methods and apparatus of the present invention will also find utility with thoracic and lumbar surgical procedures.

Not meaning to limit the methods and apparatus of the present invention to surgical procedures on the spine, it should be understood that one of skill in the surgical art may utilize them in any type of surgery where they will be of use or benefit. For example, any surgery with retraction of tissue near bone, where there is a concern regarding soft tissue slipping under the retractor and into the surgical field.

While the methods and apparatus of the present invention have been illustrated mainly by reference to human surgery, it should be understood that they may have applicability in veterinary procedures.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which this invention pertains.

I claim:

1. A surgical retractor blade comprising:
an elongated body comprising a surface suitable for abutting against soft delicate tissue, a retractor engagement end, and comprising a first anchor guide portion for receiving an anchor, and
a first anchor positioned through the first anchor guide portion, having a first end suitable for anchoring into bone.

2. The surgical retractor blade of claim 1, wherein the elongated body further comprises a portion having a slip resistant surface for contact with bone.

3. The surgical retractor blade of claim 1, wherein the anchor comprises a second end suitable for engagement with a distractor.

4. The surgical retractor blade of claim 1, wherein the anchor is selected from the group consisting of pins, screws, pegs, rods, and fasteners.

5. The surgical retractor blade of claim 1, further comprising a second anchor guide portion for receiving an anchor, and
a second anchor positioned through the second anchor guide portion and into the bone.

6. A surgical method for retracting tissue adjacent to bone comprising:
(A) making a surgical incision into tissue adjacent to bone sufficient to expose the bone;
(B) positioning a first anchorable surgical retractor blade in the incision, wherein the blade comprises:
an elongated body comprising a surface suitable for abutting against soft delicate tissue, a retractor engagement end, and
comprising a first anchor guide portion for receiving an anchor;
(C) positioning a complimentary surgical retractor blade in the incision;
(D) affixing the first anchorable and complimentary retractor blades to a retractor;
(E) operating the retractor to retract the tissue and expose the bone;
(F) positioning a first anchor through the first anchor guide portion and into the bone.

7. The surgical retractor blade of claim 1, wherein the elongated body further comprises a portion having a slip resistant surface for contact with bone.

8. The method of claim 6, wherein the anchor comprises a second end suitable for engagement with a distractor.

9. The method of claim 6, wherein the anchor is selected from the group consisting of pins, screws, pegs, rods, and fasteners.

10. The method of claim 6, wherein the first retractor blade further comprises a second anchor guide portion for receiving an anchor, and wherein step (F) of the method further comprises positioning a second anchor through the second anchor guide portion and into the bone.

11. The method of claim 6, wherein step (B) further comprises positioning a second anchorable surgical retractor blade in the incision, wherein the second blade comprises an elongated body comprising a surface suitable for abutting against soft delicate tissue, a retractor engagement end, and comprising a second anchor guide portion for receiving an anchor, and wherein step (F) further comprises positioning the first anchor through the second anchor guide portion; and wherein step (D) further comprises affixing the second anchorable retractor blade to the retractor.

12. The method of claim 6, wherein step (B) further comprises positioning a second anchorable surgical retractor blade in the incision, wherein the second blade comprises an elongated body comprising a surface suitable for abutting against soft delicate tissue, a retractor engagement end, and comprising a second anchor guide portion for receiving an anchor, and wherein step (F) further comprises positioning a second anchor through the second anchor guide portion and into the bone; and wherein step (D) further comprises affixing the second anchorable retractor blade to the retractor.

13. The method of claim 6, further comprising:
(G) affixing the first and second anchorable retractor blades to a distractor;
(H) operating the distractor to distract the bone.

14. A retractor blade kit comprising:
a first elongated body comprising a surface suitable for abutting against soft delicate tissue, a retractor engagement end, and comprising a first anchor guide portion for receiving an anchor, and
a first anchor positionable through the first anchor guide portion, having a first end suitable for anchoring into bone.

15. The surgical retractor blade kit of claim 13, further comprising:
a second elongated body comprising a surface suitable for abutting against soft delicate tissue, a retractor engagement end, and comprising a second anchor guide portion for receiving an anchor, and
wherein the first anchor is further positionable through the second anchor guide portion.

16. The surgical retractor blade kit of claim 13, wherein the elongated body further comprises a portion having a slip resistant surface for contact with bone.

17. The surgical retractor blade kit of claim 13, wherein the anchor comprises a second end suitable for engagement with a distractor.

18. The surgical retractor blade kit claim 13, wherein the anchor is selected from the group consisting of pins, screws, pegs, rods, and fasteners.

19. A surgical retractor comprising:
a first arm having a finger grip section,
a second arm having a finger grip section, and pivotally connected to the first arm,
a first surgical retractor blade supported by the first arm, comprising
an elongated body comprising a surface suitable for abutting against soft delicate tissue, and comprising an anchor guide portion for receiving an anchor, and
an anchor positioned through the anchor guide portion, having a first end suitable for anchoring into bone.

20. The surgical retractor of claim 19, wherein the elongated body further comprises a portion having a slip resistant surface for contact with bone.

21. The surgical retractor of claim 19, wherein the anchor is selected from the group consisting of pins, screws, pegs, rods, and fasteners.

22. The surgical retractor of claim 19, further comprising:
a distractor in engagement with the anchor.

23. The surgical retractor of claim 19, further comprising, a complimentary retractor blade paired with the first surgical retractor blade, supported by the second arm.

24. The surgical retractor of claim 19, further comprising:
a second surgical retractor blade supported by the first arm, comprising
an elongated body comprising a surface suitable for abutting against soft delicate tissue, and comprising an anchor guide portion for receiving an anchor, and
an anchor positioned through the anchor guide portion, having a first end suitable for anchoring into bone.

25. The surgical retractor of claim 19, further comprising, two complimentary retractor blades paired with each of the first and second surgical retractor blades, with these complimentary retractor blades supported by the second arm.

26. A method of retracting tissues adjacent a bone, using a retractor blade comprising a surface suitable for abutting against tissue, and comprising an anchor guide portion for receiving an anchor, and using an anchor positionable through the anchor guide portion, having a first end suitable for anchoring into bone, the method comprising:
(a) placing said retractor blade in a wound opening;
(b) retracting tissues surrounding the wound opening with the retractor blade;
(c) positioning the retractor blade against the bone;
(d) positioning the anchor through the anchor guide; and
(e) securing the anchor in the bone.
